(19) United States
(12) Patent Application Publication
(10) Pub. No.: US 2009/0053153 A1
(43) Pub. Date: Feb. 26, 2009

(54) Polymer composite particles containing sunscreen agent and manufacturing method thereof

(75) Inventors: Sung-Ho Lee, Seoul (KR); Jeong-Hwan Kim, Seoul (KR); Jang-Ho Park, Bucheon City (KR); Sang-Uk Kim, Siheung City (KR)

Correspondence Address: CANTOR COLBURN, LLP, 20 Church Street, 22nd Floor, Hartford, CT, 06103, US

(73) Assignee: SUNJIN CHEMICAL CO., LTD., Ansan City (KR)

(21) Appl. No.: 11/872,456

(22) Filed: Oct. 15, 2007

(30) Foreign Application Priority Data
Aug. 20, 2007 (KR) 10-2007-0083469

(51) Int. Cl. A61K 8/02; A61K 8/72; A61Q 17/04

(52) U.S. Cl. 424/60; 424/59

(57) ABSTRACT

The present invention relates to polymer composite particles containing a sunscreen agent and a manufacturing method thereof, and more particularly, to circular-shaped polymer composite particles which are a hybrid of circular-shaped polymer particles and a sunscreen agent, drastically improve dispersion stability of particles themselves, provide excellent ultraviolet absorption performance and are applicable to cosmetics, and a manufacturing method thereof.

### CROSS-REFERENCE TO RELATED APPLICATIONS

This application claims priority from Korean Patent Application No. 2007-0083469, filed on Aug. 20, 2007, in the Korean Intellectual Property Office, the disclosure of which is incorporated herein by reference.

### BACKGROUND OF THE INVENTION

1. Field of the Invention

The present invention relates to polymer composite particles containing a sunscreen agent and a manufacturing method thereof, and more particularly, to circular-shaped polymer composite particles which are a hybrid of circular-shaped polymer particles and a sunscreen agent, drastically improve dispersion stability of particles themselves, provide excellent ultraviolet absorption performance and are applicable to cosmetics, and a manufacturing method thereof.

2. Description of the Related Art

Approximately 1% of sunlight which reaches the earth's surface is generally ultraviolet rays harmful to the human body. The ultraviolet rays have a shorter wavelength than visible rays or infrared rays. As energy of light is in inverse proportion to wavelength, ultraviolet rays have larger energy than visible rays or infrared rays do. If a human is exposed to ultraviolet rays a lot, various chemical reactions may be caused by the large energy of ultraviolet rays, thereby possibly resulting in physiological changes.

As the ultraviolet rays have larger energy, they activate several chemical materials to change the structure thereof or to degrade functions. Particularly, an organic pigment which is frequently used in cosmetics or consumer products is activated by ultraviolet rays and easily loses its original functions. To this end, a sunscreen agent has long been used in blending cosmetics or consumer products.

In recent years, the radiation amount of ultraviolet rays to the Earth's surface has increased due to ozone layer depletion. Thus, the market for products which screen ultraviolet rays as their main aim has expanded greatly every year.

Most organic ultraviolet sunscreen agents have such a problem that they may cause physiological changes when used as much as needed to efficiently screen the harmful ultraviolet rays. Damage to the human body, more specifically, skin trouble is caused as the sunscreen agent penetrates the skin.

The sunscreen agent has low stability to heat and light, and is not easily dissolved in a solvent. Thus, the sunscreen agent has its limitations in usage and has a bad feeling in use. To overcome such problems, the organic sunscreen agent is encapsulated by silicon resin or glass to form a core-shell shape. In this case, the shell is broken even by weak shock so that a core material contacts the skin in a large amount. Thus, the core-shell type has many weaknesses in use, and it is not easy to adjust sizes of a capsule.

Korean Patent First Publication No. 2005-0022036 discloses a manufacturing method of encapsulating an organic sunscreen absorber by a polymer resin. In the encapsulation method, the sunscreen agent leaks out of the capsule during production processes and has harmful effects to the skin, thereby causing a bad feeling in use. Polymer material which is used as the capsule may fall apart. The sunscreen agent which is disposed in the capsule has low solvent resistance to the solvent and may leak therefrom.

Korean Patent First Publication No. 2003-0020515 discloses a manufacturing method of composite particles by dispersing an inorganic sunscreen agent for ultraviolet dispersion in a polymer. As the inorganic sunscreen agent simply disperses ultraviolet rays, it is not effective in screening ultraviolet rays. The inorganic sunscreen agent may result in milky water when used.

Thus, it is necessary to develop a technology to use the sunscreen agent stably and efficiently.

### SUMMARY OF THE INVENTION

Accordingly, it is an aspect of the present invention to provide polymer composite particles which are a hybrid of a sunscreen agent having many limitations in use and a circular-shaped polymer material, minimize skin irritation, are used efficiently and stably and provide excellent sunscreen effect, and a manufacturing method thereof.

Also, it is another aspect of the present invention to provide a sunscreen cosmetic composition which contains polymer composition particles having the sunscreen agent, as an active component.

Additional aspects and/or advantages of the present invention will be set forth in part in the description which follows and, in part, will be obvious from the description, or may be learned by practice of the present invention.

The foregoing and/or other aspects of the present invention are also achieved by providing polymer composite particles comprising a) polymer particles which polymerize a radical polymerizable monomer and a multifunctional cross-linking monomer; and b) polymer particles which are mixed with a sunscreen agent.

The foregoing and/or other aspects of the present invention are also achieved by providing a manufacturing method of polymer composite particles, the manufacturing method comprising a) dissolving and dispersing a sunscreen agent in a radical polymerizable monomer or a multifunctional cross-linking monomer and adding an initiator thereto; b) emulsifying the dispersion containing the sunscreen agent in a solvent under a presence of a dispersion stabilizer to form droplets; c) polymerizing the droplets to manufacture polymer composite particles; and d) cleansing and drying the manufactured polymer composite particles.

The foregoing and/or other aspects of the present invention are also achieved by providing a sunscreen cosmetic composition which contains polymer composite particles containing a sunscreen agent, as an active component.

### BRIEF DESCRIPTION OF THE DRAWINGS

The above and/or other aspects of the present invention will become apparent and more readily appreciated from the following description of the embodiments, taken in conjunction with the accompanying drawings of which:

FIG. 1 illustrates an electron microscopic picture of polymer composite particles which contain a sunscreen agent manufactured according to an exemplary embodiment 1;

FIG. 2 illustrates an electron microscopic picture of polymer composite particles which contain a sunscreen agent manufactured according to an exemplary embodiment 2;

FIG. 3 illustrates an electron microscopic picture of polymer composite particles which contain a sunscreen agent manufactured according to an exemplary embodiment 3;

FIG. 4 illustrates an electron microscopic picture of polymer composite particles which contain a sunscreen agent manufactured according to an exemplary embodiment 4; and

FIG. 5 illustrates an electron microscopic picture of polymer composite particles which contain a sunscreen agent manufactured according to an exemplary embodiment 5.

### DETAILED DESCRIPTION OF EXEMPLARY EMBODIMENT

Hereinafter, exemplary embodiments of the present invention will be described with reference to accompanying drawings, wherein like numerals refer to like elements and repetitive descriptions will be avoided as necessary.

Polymer composition particles according to the present invention include a) polymer particles which polymerize a radical polymerizable monomer and a multifunctional cross-linking monomer; and b) polymer composite particles which are mixed with a sunscreen agent.

The radical polymerizable monomer according to the present invention is copolymerizable with a vinyl cross-linking monomer, and does not have particular limitation. More specifically, the radical polymerizable monomer includes an aromatic vinyl compound such as styrene, methylstyrene, ethylstyrene, fluorostyrene, chlorostyrene, vinyltoluene; an acrylic vinyl monomer such as methyl (metha)acrylate, ethyl (metha)acrylate, butyl(metha)acrylate, octyl(metha)acrylate, stearyl(metha)acrylate, benzyl(metha)acrylate, glycidyl(metha)acrylate, fluoroethylacrylate, etc.; and a cyanide vinyl monomer such as (metha)acrylonitrile, etc. The foregoing monomers may be used by themselves, or at least two of the monomers may be mixed with each other.

The multifunctional cross-linking monomer is used to cross-link particles. The multifunctional cross-linking monomer may be copolymerizable with the radical polymerizable monomers. More specifically, the multifunctional cross-linking monomer includes divinylbenzene, ethyleneglycoldimethaacrylate, diethyleneglycolmethaacrylate, triethyleneglycoldimethaacrylate, trimethylenepropanetrimethaacrylate, 1,3-butanediolmethacrylate, 1,6-hexanedioldimethacrylate, allyl(metha)acrylate, tri(metha)acrylate, etc., which may be used by themselves or at least two of them may be mixed with each other.

Preferably, the content ratio of the radical polymerizable monomer and the multifunctional cross-linking monomer is 5 to 95% by weight: 95 to 5% by weight. More preferably, the content ratio of the radical polymerizable monomer and the multifunctional cross-linking monomer is 30 to 70% by weight: 70 to 30% by weight. If the content ratio thereof ranges as described above, the physical properties of the manufactured polymer composite particles improve. Also, the particle size may be properly adjusted to apply to cosmetics. Particularly, if the content of the multifunctional cross-linking monomer is less than 5% by weight, the manufactured particles have lowered solvent resistance, heat resistance and mechanical properties.

The b) sunscreen agent may include a chemical sunscreen agent. The sunscreen agent is classified into a chemical sunscreen agent (sunscreen absorber) blocking ultraviolet rays by absorption and a physical sunscreen agent blocking ultraviolet rays by reflection and dispersion. The sunscreen agent according to the present invention includes a chemical sunscreen agent. Also, the sunscreen agent according to the present invention may include an organic/inorganic chemical sunscreen agent, and more preferably an organic sunscreen agent. More specifically, the organic sunscreen agent includes p-aminobenzoic acid (PABA) derivatives, cinnamate derivatives, salicylic acid derivatives, benzophenone, dibenzole methane, antranilate, camphor derivatives, methylene bis-benzotriazoyl tetramethylbutyl phenol, bis-ethylhexyloxyphenol methoxyphenyl triazine, etc. The organic sunscreen agent comprises at least one or more of them (p-aminobenzoic acid (PABA) derivatives, cinnamate derivatives, salicylic acid derivatives, benzophenone, dibenzole methane, antranilate, camphor derivatives, methylene bis-benzotriazoyl tetramethylbutyl phenol, bis-ethylhexyloxyphenol methoxyphenyl triazine).

The foregoing sunscreen agents may be used by themselves, or at least two of them may be mixed with each other. Preferably, the sunscreen agent is included as much as 0.1 to 200% by weight with respect to 100% by weight of the sum total of the radical polymerizable monomer and the multifunctional cross-linking monomer.

The polymer composite particles may be manufactured in various sizes. Preferably, an average particle diameter of the polymer composite particles ranges from 0.01 μm to 1000 μm.

The polymer composite particles according to the present invention may be manufactured by including a) an operation of dissolving and dispersing a sunscreen agent in the radical polymerizable monomer or the multifunctional cross-linking monomer and adding an initiator thereto; b) an operation of emulsifying the dispersion containing the sunscreen agent in a solvent under a presence of a dispersion stabilizer to form droplets; c) an operation of polymerizing the droplets and manufacturing polymer composite particles; and d) an operation of cleansing and drying the manufactured polymer composite particles.

The dissolution and dispersion in the operation a) according to the present invention may include typical methods. The initiator added may vary as long as it forms free radicals. More specifically, the initiator added may include peroxide series including benzoylperoxide, laurylperoxide, cumene hydroperoxide, methylethylketoneperoxide, t-butylhydroperoxide, etc.; azo compounds including 2,2′-azobisisobutylonitrile, 2,2′-azobis-2,4-dimethylvaleronitrile, 2,2′-azobis-2-methylisobutylonitrile, or mixtures thereof. The initiator may be used as much as 0.1 to 10% by weight, preferably 0.5 to 5% by weight with respect to 100% by weight of the sum total of the radical polymerizable monomer and the multifunctional cross-linking monomer. If the content of the initiator ranges as described above, yield improves, heat is less generated in a polymerization process, and the size of the particles is adjusted without difficulty.

In the operation b), the dispersion manufactured in the operation a) is emulsified by the solvent under the presence of the dispersion stabilizer to form the droplets. The droplets may be formed by using typical methods.

The dispersion stabilizer is used to prevent condensation and precipitation of the particles formed by the polymerization reaction. More specifically, the dispersion stabilizer includes cellulose derivatives including methylcellulose, ethylcellulose, hydroxypropylcellulose, etc.; polyvinylalcohol, polyvinylmethylether, polyacrylic acid, polyvinylacetate or a copolymer of polyvinylpyrrolidone and vinylacetate; colloidal silica, etc. Preferably, the dispersion stabilizer is included as much as 0.1 to 20% by weight, and more preferably 0.5 to 10% by weight with respect to 100% by weight of the sum total of the radical polymerizable monomer and the multifunctional cross-linking monomer. If the concentration of the dispersion stabilizer is lower than 0.1% by weight, it becomes condensed. If the concentration of the dispersion stabilizer exceeds 20% by weight, it becomes gel and sub reactions may occur.

The solvent which is used to emulsify the dispersion stabilizer includes ion exchange water, distilled water, alcohol, ether alcohol, etc. The foregoing solvents may be used by themselves or at least two of them may be mixed with each other.

The polymerization in the operation c) may be performed by typical methods used by those skilled in the art. More specifically, the polymerization methods include suspension polymerization, emulsion polymerization, solution polymerization or mass polymerization. Particularly, the suspension polymerization is preferable in the present invention. Preferably, the polymerization is performed at 50° C. to 90° C. for 5 to 50 hours to have higher conversion rate. Here, if agitation speed is too high in the polymerization reaction, the condensation may occur, or the size of the particles may not be adjusted easily as the size of the particles is changed. Thus, it is required to maintain proper agitation speed.

In the operation d), the polymer composite particles manufactured in the operation c) are cleansed and dried.

The particles are cleansed to reduce the amount of an unreacted monomer existing in the particles after filtering the composite particles, remove impurities such as the initiator remaining after the polymerization reaction, thereby preventing coloring due to the impurities even if temperatures are raised during the drying operation. The polymer composite particles may be cleansed by alcohol (methylalcohol, ethylalcohol, butylalcohol, amylalcohol, octylalcohol or benzylalcohol, etc.), polyhydric alcohol (ethylene glycol, propylene glycol, glycerin, etc.), aqueous solution thereof or water. Preferably, the polymer composite particles may be cleansed by using ethanol, methyl alcohol, ethyl alcohol, propyl alcohol, isopropyl alcohol, butyl alcohol, pentyl alcohol, hexylalcohol, heptyl alcohol or octyl alcohol. Here, the aqueous solution may be used as much as 1 to 30% by weight of the cleansing solution. If the aqueous solution is used in the range described above, the removing efficiency of the impurities may improve.

The polymer composite particles may be dried in a vacuum at a temperature lower than a glass transition temperature in consideration of the glass transition temperature of the particles which have removed the impurities therefrom and have been cleansed.

Polymethylmethaacrylate beads which are manufactured by the foregoing operations according to the present invention may further have a classification operation to adjust distribution of the particle sizes. Here, the classification may be performed by a typical method. More specifically, the beads may be classified by sieve and by adjusting feeding speed, the number of rotation and airflow of a typical classifier, thereby controlling the distribution of sizes of the beads.

The present invention provides a sunscreen cosmetic composition which contains polymer composition particles having the sunscreen agent, as an active component.

The polymer composite particles containing the sunscreen agent may be added to various formulations of the cosmetic composition. Preferably, the polymer composite particles are added to as much as 0.01 to 50 wt % with respect to the total weight of the composition. If the content of the polymer composite particles is less than 0.01 wt %, the sunscreen effect is very low. If the content of the polymer composite particles exceeds 50 wt %, problems may arise in the formulations of the cosmetic composition. The cosmetic compositions include cosmetic or personal care formulation, which includes skin care, sun care, make-up, hair care and body care. They include not only general sun screen products but also all kinds of cosmetic and personal care formulation that have the function of sun screening.

Hereinafter, preferable exemplary embodiments are described to help understanding of the present invention. The exemplary embodiments exemplify the present invention, and the scope of the present invention is not limited thereto.

### Exemplary Embodiment 1

Thirteen percent by weight of methylmethacrylate as the radical polymerizable monomer, 1.3% by weight of ethyleneglycoldimethacrylate as the multifunctional cross-linking monomer, 0.2% by weight of 2,2′-azobis-2,4-dimethylvaleronitrile as the initiator, 4.5% by weight of butylmethoxydibenzoylmethane as the sunscreen agent, 0.3% by weight of polyvinylalcohol as the dispersion stabilizer, 80.7% by weight of ion exchange water as the solvent are provided with respect to 100% by weight of total reactants in a detachable chemical reactor having an agitator. The foregoing mixed materials are emulsified by an emulsifier (homogenizer) at 8,000 rpm for ten minutes. After the emulsification process, the temperatures are raised to 60° C. The foregoing materials are polymerized at 60° C. for 20 hours. The obtained polydisperse polymer particles are dehydrated by a decompression filter, cleansed by ethanol several times, and dried in a vacuum, thereby forming a final product.

### Exemplary Embodiment 2

The exemplary embodiment 2 is the same as the exemplary embodiment 1 except that 6.5% by weight of butylmethoxydibenzoylmethane is used as a sunscreen agent.

### Exemplary Embodiment 3

The exemplary embodiment 3 is the same as the exemplary embodiment 1 except that 6.5% by weight of octylmethoxycinnamate is used as a sunscreen agent instead of butylmethoxydibenzoylmethane.

### Exemplary Embodiment 4

The exemplary embodiment 4 is the same as the exemplary embodiment 1 except that a mixture of 4.5% by weight of butylmethoxydibenzoylmethane and 4.5% by weight of octylmethoxycinnamate is used as a sunscreen agent instead of butylmethoxydibenzoylmethane.

### Exemplary Embodiment 5

The exemplary embodiment 5 is the same as the exemplary embodiment 1 except that a mixture of 6.5% by weight of butylmethoxydibenzoylmethane and 6.5% by weight of octylmethoxycinnamate is used as a sunscreen agent instead of butylmethoxydibenzoylmethane.

### Experimental Embodiment 1

### Experiment on Properties and Sunscreen Performance

### Formulation Example 1 and Comparative Embodiment 1

A W/O emulsion sunscreen cosmetic is manufactured by using the polymer particles manufactured according to the exemplary embodiment 1 to check the sunscreen performance of the polymer composite particles according to the exemplary embodiment 1. The basic composition is shown in Table 1.

TABLE 1

Content (% by weight)

| | Formulation | Comparative |
|---|---|---|
| Composition | example 1 | embodiment 1 |
| Alkylbenzoate | 42.0 | 42.0 |
| Silicon emulsifier(KF6027) | 4.0 | 4.0 |
| Bentonite | 2.0 | 2.0 |
| Polymer composite particles | 12.0 | — |
| obtained according to | | |
| exemplary embodiment 1 | | |
| Polymethylmethacrylate | — | 2.8 |
| powder | | |
| Butylmethoxydibenzoylmethane | — | 9.2 |
| Distilled water | 20.0 | 20.0 |

### Formulation Example 2 and Comparative Embodiment 2

A W/O emulsion sunscreen cosmetic is manufactured by using the composite particles manufactured according to the exemplary embodiment 2. The basic composition is shown in Table 2.

TABLE 2

Content (% by weight)

| | Formulation | Comparative |
|---|---|---|
| Composition | example 2 | embodiment 2 |
| Alkylbenzoate | 42.0 | 42.0 |
| Silicon emulsifier (KF6027) | 4.0 | 4.0 |
| Bentonite | 2.0 | 2.0 |
| Polymer composite particles | 12.0 | — |
| obtained according to | | |
| exemplary embodiment 2 | | |
| Polymethylmethacrylate | — | 3.75 |
| powder | | |
| Butylmethoxydibenzoylmethane | — | 8.25 |
| Distilled water | 20.0 | 20.0 |

### Formulation Example 3 and Comparative Embodiment 3

A W/O emulsion sunscreen cosmetic is manufactured by using the composite particles manufactured according to the exemplary embodiment 3. The basic composition is shown in Table 3.

TABLE 3

Content (% by weight)

| | Formulation | Comparative |
|---|---|---|
| Composition | example 3 | embodiment 3 |
| Alkylbenzoate | 42.0 | 42.0 |
| Silicon emulsifier (KF6027) | 4.0 | 4.0 |
| Bentonite | 2.0 | 2.0 |
| Polymer composite particles | 12.0 | — |
| obtained according to | | |
| exemplary embodiment 3 | | |
| Polymethylmethacrylate | — | 3.75 |
| powder | | |
| octylmethoxycinnamate | — | 8.25 |
| Distilled water | 20.0 | 20.0 |

### Formulation Example 4 and Comparative Embodiment 4

A W/O emulsion sunscreen cosmetic is manufactured by using the composite particles manufactured according to the exemplary embodiment 4. The basic composition is shown in Table 4.

TABLE 4

Content (% by weight)

| | Formulation | Comparative |
|---|---|---|
| Composition | example 4 | embodiment 4 |
| Alkylbenzoate | 42.0 | 42.0 |
| Silicon emulsifier (KF6027) | 4.0 | 4.0 |
| Bentonite | 2.0 | 2.0 |
| Polymer composite particles | 12.0 | — |
| obtained according to | | |
| exemplary embodiment 4 | | |
| Polymethylmethacrylate | — | 7.47 |
| powder | | |
| Butylmethoxydibenzoylmethane | — | 2.265 |
| octylmethoxycinnamate | — | 2.265 |
| Distilled water | 20.0 | 20.0 |

### Formulation Example 5 and Comparative Embodiment 5

A W/O emulsion sunscreen cosmetic is manufactured by using the composite particles manufactured according to the exemplary embodiment 5. The basic composition is shown in Table 5.

TABLE 5

Content (% by weight)

| | Formulation | Comparative |
|---|---|---|
| Composition | example 4 | embodiment 4 |
| Alkylbenzoate | 42.0 | 42.0 |
| Silicon emulsifier (KF6027) | 4.0 | 4.0 |
| Bentonite | 2.0 | 2.0 |
| Polymer composite particles | 12.0 | — |
| obtained according to | | |
| exemplary embodiment 5 | | |
| Polymethylmethacrylate | — | 6.29 |
| powder | | |
| Butylmethoxydibenzoylmethane | — | 2.855 |
| octylmethoxycinnamate | — | 2.855 |
| Distilled water | 20.0 | 20.0 |

The particle size and shape of the final product according to the exemplary embodiments 1 to 5 are measured by using a particle size analyzer and a scanning electron microscopy. The sunscreen performance of the W/O emulsion manufactured according to the formulation examples 1 to 5 is measured by an SPF (sun protection factor) 290 analyzer manufactured by Optometrix (U.S.). The measurement result is shown in Table 6.

According to the measurement result, circular-shaped polymer particles having an average particle diameter of 4.7 μm are measured. The fine powder is added as much as 12% to manufacture the W/O cream and measure the sunscreen performance thereof. According to the measurement result, the W/O cream has very high protection factors (PF) values and sun protection factor (SPF) values.

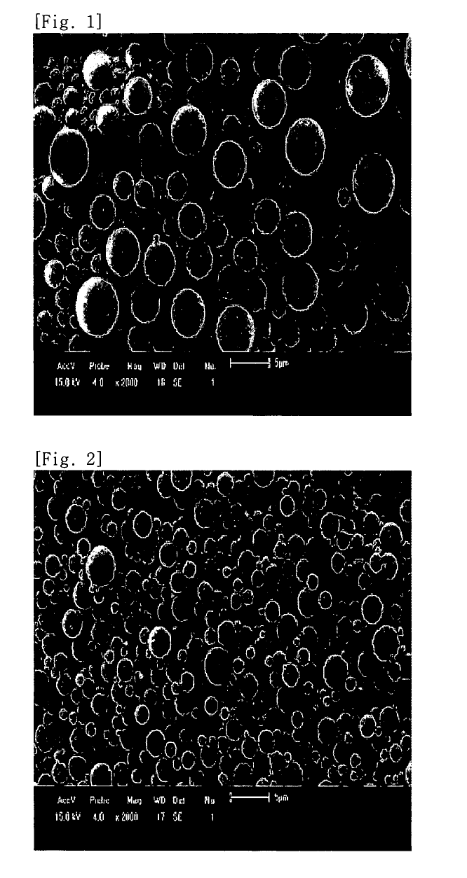

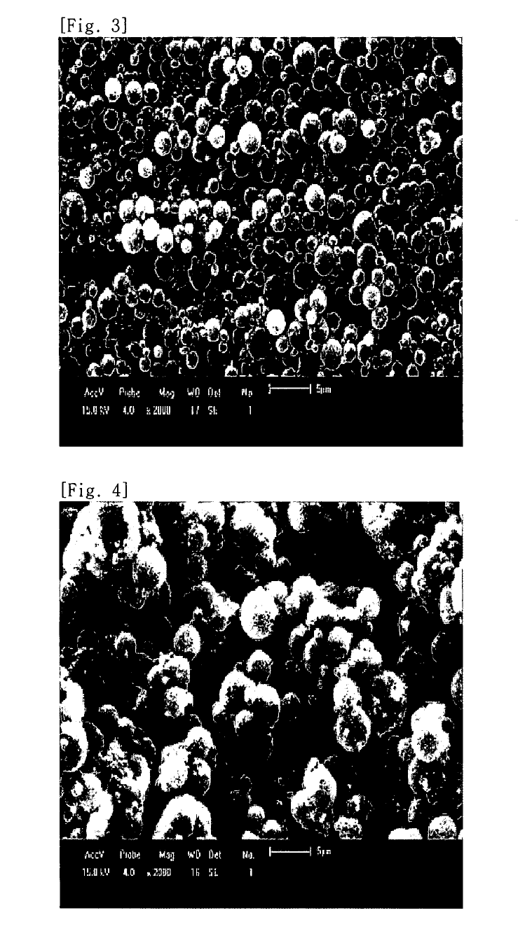

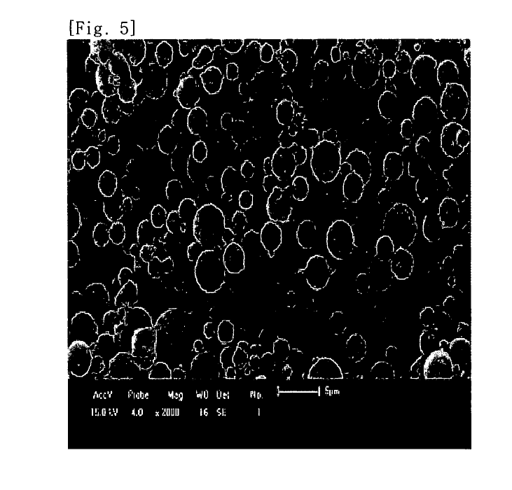

Electron microscopic pictures of the polymer particles containing the sunscreen agent manufactured according to the exemplary embodiments 1 to 5 are shown in FIGS. 1 to 5.

TABLE 6

| | Form. | Com. | Form. | Com. | Form. | Com. | Form. | Com. | Form. | Com. |
|---|---|---|---|---|---|---|---|---|---|---|
| | Exam. 1 | Embo. 1 | Exam. 2 | Embo. 2 | Exam. 3 | Embo. 3 | Exam. 4 | Embo. 4 | Exam. 5 | Embo. 5 |
| Weight ratio (%) | 12 | — | 12 | — | 12 | — | 12 | — | 12 | — |
| of polymer | | | | | | | | | | |
| composite | | | | | | | | | | |
| particles | | | | | | | | | | |
| containing | | | | | | | | | | |
| sunscreen agent | | | | | | | | | | |
| Weight ratio (%) | 2.8 | 2.8 | 3.75 | 3.75 | 3.75 | 3.75 | 4.53 | 4.53 | 5.71 | 5.71 |
| of sunscreen | | | | | | | | | | |
| agent from | | | | | | | | | | |
| sunscreen | | | | | | | | | | |
| cosmetics | | | | | | | | | | |
| Average particle | 4.7 | — | 2.7 | — | 2.1 | — | 2.2 | — | 2.4 | — |
| diameter (μm) | | | | | | | | | | |
| SPF | 10.50 | 22.2 | 14.43 | 25.51 | 10.20 | 10.64 | 20.64 | 16.94 | 21.39 | 19.18 |

### Experimental Embodiment 2

### Skin Irritation Test

A skin patch test is performed to 20 testers to test skin irritation by using the sunscreen cosmetic according to the formulation example 5 and comparative embodiment 5 having the best sunscreen effect among the sunscreen cosmetic compositions manufactured according to the formulation examples 1 to 5. The test result is shown in Table 7.

TABLE 7

| | Product of | Product of |
|---|---|---|
| | formulation | comparative |
| | example 5 | embodiment 5 |
| +++ | 0 | 7 |
| ++ | 1 | 5 |
| + | 5 | 4 |
| ± | 7 | 4 |
| − | 7 | 0 |
| Total testers | 20 | 20 |

* Reference
+++: strong trouble,
++: weak trouble
+: slight trouble,
±: normal
−: negative

As shown in Table 7, 12 testers have skin trouble to the sunscreen cosmetic according to the comparative embodiment 5, and 4 testers have slight skin trouble thereto. Meanwhile, one tester has weak skin trouble and five testers have slight skin trouble to the sunscreen cosmetic according to the formulation example 5.

### Formulation Example 6

A W/O emulsion sunscreen cosmetic is manufactured by using the polymer composite particles manufactured according to the exemplary embodiment 5. The basic composition is shown in Table 8.

TABLE 8

| | Content (% by weight) |
|---|---|
| Composition | Formulation example 6 W/O |
| Microcrystalline wax | 3.0 |
| Liquid paraffin | 7.0 |
| Silicon oil | 6.0 |
| Cyclopentasilicon | 6.0 |
| Paraben | 0.1 |
| cetyldimethiconecopolyol | 2.0 |
| Polymer composite particles | 8.33 |
| obtained according to | |
| exemplary embodiment 5 | |
| Butylmethoxydibenzoylmethane | — |
| octylmethoxycinnamate | — |
| Ethylhexylmethoxycinnamate | 7.0 |
| Butyleneglycol | 5.0 |
| water | 61.4 |

### Formulation Example 7

A W/O emulsion sunscreen cosmetic is manufactured by using the polymer composite particles manufactured according to the exemplary embodiment 5. The basic composition is shown in Table 9.

TABLE 9

| | Content (% by weight) |
|---|---|
| Composition | Formulation example 7 W/O |
| Bees wax | 2.0 |
| Stearyl alcohol | 1.5 |
| Stearic acid | 0.5 |
| Squalane | 10.0 |
| Polyoxyethylene ethylether | 1.0 |
| Propyleneglycol monostearate | 3.0 |
| ethylhexylmethoxycinnamate | 7.0 |
| Paraben | 0.1 |
| Polymer composite particles | 8.33 |
| obtained according to | |
| exemplary embodiment 5 | |
| Butylmethoxydibenzoylmethane | — |
| octylmethoxycinnamate | — |
| Propylene glycol | 8.0 |
| glycerine | 4.0 |
| Triethylamine | 59.4 |

### EFFECT OF INVENTION

Polymer composite particles which contain a sunscreen agent according to the present invention are a hybrid of a sunscreen agent having many limitations in use and a circular-shaped polymer material, minimize skin irritation, are used efficiently and stably and provide excellent sunscreen effect.

The size of the polymer composite particles according to the present invention is adjusted from 0.01 μm to 1,000 μm without difficulty. The polymer composite particles may contain a physical sunscreen agent, thereby providing high applicability.

Thus, the polymer composite particles containing the sunscreen agent according to the present invention add the chemical sunscreen agent (sunscreen absorber) to the circular-shaped polymer material to address the problems of the chemical sunscreen agent at the timing of requiring a sunscreen agent having high sunscreen performance due to ozone layer depletion. The chemical sunscreen agent has such problems that it is harmful to the human body such as contact dermatitis, is not easily dissolved to have a limited usage range, has a bad feeling in use, and does not have good storage stability while providing good sunscreen performance.

Although a few exemplary embodiments of the present invention have been shown and described, it will be appreciated by those skilled in the art that changes may be made in these exemplary embodiments without departing from the principles and spirit of the invention, the scope of which is defined in the appended claims and their equivalents.

What is claimed is:

1. Polymer composite particles, comprising:
a) polymer particles which polymerize a radical polymerizable monomer and a multifunctional cross-linking monomer; and
b) polymer particles which are mixed with a sunscreen agent.

2. The polymer composite particles according to claim 1, wherein a content ratio of the a) radical polymerizable monomer and the multifunctional cross-linking monomer is 5 to 95% by weight: 95 to 5% by weight.

3. The polymer composite particles according to claim 1, wherein the b) sunscreen agent is included as much as 0.1 to 200% by weight with respect to 100% by weight of a sum total of the radical polymerizable monomer and the multifunctional cross-linking monomer.

4. The polymer composite particles according to claim 1, wherein the radical polymerizable monomer comprises at least one an aromatic vinyl compound such as styrene, methylstyrene, ethylstyrene, fluorostyrene, chlorostyrene, vinyltoluene; an acrylic vinyl monomer such as methyl (metha)arcylate, ethyl (metha)acrylate, butyl(metha)acrylate, octyl(metha)acrylate, stearyl(metha)acrylate, benzyl(metha)acrylate, glycidyl(metha)acrylate, fluoroethylacrylate; and a cyanide vinyl monomer such as (metha)acrylonitrile; and mixtures thereof.

5. The polymer composite particles according to claim 1, wherein the multifunctional cross-linking monomer comprises at least one of divinylbenzene, ethyleneglycoldimethaacrylate, diethyleneglycolmethaacrylate, triethyleneglycoldimethaacrylate, trimethylenepropanetrimethaacrylate, 1,3-butanediolmethacrylate, 1,6-hexanedioldimethacrylate, allyl(metha)acrylate, tri(metha)acrylate; and mixtures thereof.

6. The polymer composite particles according to claim 1, wherein the b) sunscreen agent comprises at least one of p-aminobenzoic acid (PABA) derivatives, cinnamate derivatives, salicylic acid derivatives, benzophenone, dibenzole methane, antranilate, camphor derivatives, methylene bis-benzotriazoyl tetramethylbutyl phenol, bis-ethylhexyloxyphenol methoxyphenyl triazine.

7. The polymer composite particles according to claim 1, wherein an average particle diameter of the polymer composite particles ranges from 0.01 μm to 1,000 μm.

8. The polymer composite particles according to claim 1, wherein the polymer composite particles are used for sunscreen.

9. A manufacturing method of polymer composite particles, the manufacturing method comprising:
a) dissolving and dispersing a sunscreen agent in a radical polymerizable monomer or a multifunctional cross-linking monomer and adding an initiator thereto;
b) emulsifying the dispersion containing the sunscreen agent in a solvent under a presence of a dispersion stabilizer to form droplets;
c) polymerizing the droplets to manufacture polymer composite particles; and
d) cleansing and drying the manufactured polymer composite particles.

10. The manufacturing method according to claim 9, wherein the initiator in the operation a) comprises at least one of peroxide series including benzoylperoxide, laurylperoxide, cumene hydroperoxide, methylethylketoneperoxide, t-butylhydroperoxide; azo compounds including 2,2′-azobisisobutylonitrile, 2,2′-azobis-2,4-dimethylvaleronitrile, 2,2′-azobis-2-methylisobutylonitrile, and is included as much as 0.1 to 10% by weight with respect to 100% by weight of a sum total of the radical polymerizable monomer and the multifunctional cross-linking monomer.

11. The manufacturing method according to claim 9, wherein the dispersion stabilizer in the operation b) comprises at least one of cellulose derivatives including methylcellulose, ethylcellulose, hydroxypropylcellulose; polyvinylalcohol, polyvinylmethylether, polyacrylic acid, polyvinylacetate or a copolymer of polyvinylpyrrolidone and vinylacetate; and colloidal silica, and is included as much as 0.1 to 20% by weight with respect to 100% by weight of a sum total of the radical polymerizable monomer and the multifunctional cross-linking monomer.

12. A sunscreen cosmetic composition which contains the polymer composite particles according to claim 1 as an active component.

13. The sunscreen cosmetic composition according to claim 12, wherein the polymer composite particles containing a sunscreen agent is included as much as 0.01 to 50% by weight with respect to a total weight of the composition.
